(19) United States
(12) Patent Application Publication
(10) Pub. No.: US 2006/0104858 A1
(43) Pub. Date: May 18, 2006

(54) Hydroxyl free radical-induced decontamination of airborne spores, viruses and bacteria in a dynamic system

(76) Inventors: Richard S Potember, Dayton, MD (US); Wayne A Bryden, Ellicott City, MD (US)

Correspondence Address: Francis A Cooch;Deputy General Counsel, The Johns Hopkins University Applied Physics Lab, 11100 Johns Hopkins, Laurel, MD, 20723-6099, US

(21) Appl. No.: 10/541,112

(22) PCT Filed: Jun. 18, 2003
(86) PCT No.: PCT/US03/19075

§ 371 Date: Jun. 30, 2005

Related U.S. Application Data
(60) Provisional application No. 60/438,287, filed on Jan. 6, 2003.

(51) Int. Cl. A61L 9/015; A61L 9/14; A61L 9/20

(52) U.S. Cl. 422/4; 422/121; 422/123

(57) ABSTRACT

A method and apparatus is described for neutralizing airborne pathogens and chemical toxins in ventilated air, and in heating or air conditioning systems. The pathogen-chemical toxin neutralization system is effective against a wide spectrum of pathogens and toxins, it incorporates commercially available components, and it can be readily integrated into commercial HVAC systems where it decontaminates large volumes of ventilated air in real time without any chemical reagents. The system has a flow-through reaction chamber (101) that contains a UV light source (106) that emits short intense flashes of broad-spectrum UV light, a source aqueous hydrogen peroxide that can be a reservoir or a hydrogen peroxide generator (106), and optionally a source of ozone. The interaction of UV light and hydrogen peroxide generates hydroxyl radicals that neutralize pathogens and chemical toxins as they pass through the reaction chamber (101) in real time. The pathogens that can be neutralized by this system include bacteria, viruses, spores, fungi and parasites.

### CROSS-REFERENCE TO RELATED APPLICATION

This application claims the benefit of prior filed co-pending U.S. Provisional Application Ser. No. 60/438,287, filed Jan. 6, 2003, which is incorporated herein by reference as if fully set forth herein under 35 U.S.C. Section 119(e).

This application is also related to U.S. Ser. No. 10/257,196 filed on Oct. 9, 2002 (hereafter “Potember”) which is incorporated herein by reference as if fully set forth herein.

### BACKGROUND OF THE INVENTION

1. Field of the Invention

The present invention relates to a new, safe, effective method for neutralizing or destroying a wide range of airborne pathogens (spores, bacteria and viruses) and chemical toxins in commercial HVAC air handling systems.

2. Description of the Related Art

It is critical to develop rapid, effective, and safe (nontoxic and not corrosive) technologies for neutralizing airborne pathogens and chemical toxins to protect civilian and military facilities from a chemical or biological attack. Within this area, there is a special need to protect domed stadiums, subways, and enclosed facilities (buildings and command centers that may contain extremely sensitive equipment). This effort is a key to eliminate the threat of biological and chemical weapons in the planning and conduct of US military operations. While no defense can stop an adversary from unleashing such weapons, a sufficiently robust array of defenses, countermeasures and deterrents will reduce the damage resulting from biological and chemical weapons used in a particular operation.

There is also a great need to remove airborne pathogens from air handling systems in hospitals and on airplanes where the transmission of respiratory infections in indoor environments represents a major public health concern for which engineering alternatives are limited. Evidence for the dissemination of respiratory diseases inside buildings, and specifically by ventilation systems, exists in the epidemiological data. The risk to patients of becoming infected with Staphylococcus, one of the most common and deadly infections associated with prolonged hospital stay, is significant.

To accomplish these goals, a pathogen-toxin neutralization technology is needed that can destroy a wide range of pathogens (spores, bacteria, and viruses) and chemical toxins in air in real time as the air moves through an HVAC system without introducing contamination into the air handling system. The neutralization of airborne biological and chemical toxins is a very difficult problem to solve because, to be useful, it must work in real time, and handle large volumes of moving air.

In Potember, a UV/ozone pathogen neutralization system was disclosed based on the discovery that irradiating ozone with high intensity, broad spectrum UV light in the presence of water generates highly reactive ozone intermediates and free radicals that destroy a wide range of airborne pathogens. The ozone intermediates and free radicals were more effective at neutralizing pathogens than ozone or UV light alone. Massive amounts of airborne Erwinia herbicola vegetative bacteria and MS2 virus introduced into the UV/ozone system were reduced to undetectable levels. However, while the system was able to destroy approximately one to two orders of magnitude of extremely high levels of airborne Bacillus globii spores, an anthrax stimulant, it was not able to reduce the total population of airborne spores to undetectable levels. Therefore, there is a need for a pathogen neutralization system with increased efficiency for neutralizing airborne bacterial spores in real time.

The past approaches described in this section could be pursued, but are not necessarily approaches that have been previously conceived or pursued. Therefore, unless otherwise indicated herein, the approaches described in this section are not prior art to the claims in this application and are not admitted to be prior art by inclusion in this section.

### SUMMARY OF THE INVENTION

A system for neutralizing airborne pathogens or chemical toxins is disclosed. The system has a flow-through reaction chamber with a chamber air inlet at a first end of the reaction chamber to admit air contaminated with pathogens, and a chamber air outlet at a second end of the reaction chamber to release decontaminated air, and defining between the air inlet and air outlet a passageway. The system further includes a supply of aqueous hydrogen peroxide connected to a conduit for introducing aqueous hydrogen peroxide into the reaction chamber, and an ultraviolet light source for introducing UV light into the reaction chamber.

In an embodiment of this aspect, the aqueous hydrogen peroxide supply is a hydrogen peroxide generator connected to a water supply and a source of electricity. In another embodiment, the aqueous hydrogen peroxide supply is a reservoir of aqueous hydrogen peroxide that can be located inside or outside the reaction chamber. In some embodiments, the conduit includes a nozzle disposed inside the reaction that releases the aqueous hydrogen peroxide as a spray, mist or vapor. To provide additional surface area, on which the neutralization process can occur, some embodiments of the system optionally contain a porous matrix such as metal foam made of aluminum, copper, silver, or metal oxides. In some embodiments, the neutralization system includes an ozone supply in addition to the aqueous hydrogen peroxide supply and a conduit for introducing ozone into the reaction chamber that can optionally be a nozzle disposed inside the reaction chamber. In some embodiments the ozone supply is an ozone generator or a reservoir of ozone. In another embodiment, the aqueous hydrogen peroxide supply is connected by a conduit to the ozone supply so that ozone passes from the ozone supply into the aqueous hydrogen peroxide supply thereby forming a mixture of hydrogen peroxide, water and ozone that can be sprayed into the reaction chamber.

In some embodiments of the aspect, the reaction chamber includes a solid support that is made of or coated with ozone removal catalysts. The solid support can also be made of or coated with compounds that adsorb or neutralize pathogens or chemical toxins or both. In the various embodiments, the neutralization system is configured for operation in a continuous mode or is activated upon demand. The system optionally includes a fan. Embodiments include an UV light source in the reaction chamber that emits high intensity UV light. In an embodiment of the aspect the UV light has a wavelength in a range from about 100 to about 350 nm.

In another aspect of the invention, methods of neutralizing airborne pathogens or chemical toxins include the steps of introducing air contaminated with pathogens or chemical toxins or both into a flow-through reaction chamber. Aqueous hydrogen peroxide is introduced into the flow-through reaction chamber to form a mixture of contaminated air and aqueous hydrogen peroxide. The mixture is irradiated with ultraviolet light thereby neutralizing the airborne pathogens or chemical toxins. The decontaminated air is released from the reaction chamber. In another embodiment of this aspect, the method further includes introducing ozone into the reaction chamber to form a mixture of ozone, contaminated air and aqueous hydrogen peroxide that is irradiated inside the reaction chamber with UV light. In an embodiment of this aspect, a concentration of hydrogen peroxide in the flow through reaction chamber is maintained at a level in a range from about 1% to about 50%; in another embodiment the concentration is maintained at a level in a range from about 1% to about 25%. In some embodiments that include introducing ozone into the reaction chamber, the concentration of ozone in the reaction chamber is maintained at a level in a range of from about 0.01 ppm to about 1000 ppm, or in a range from about 0.1 ppm (part per million) to about 1000 ppm.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a block diagram of an embodiment of the UV/H₂O₂ neutralization system having the hydrogen peroxide (H₂O₂) generator 104 disposed inside the flow-through reaction chamber 101.

FIG. 2 is a block diagram of an embodiment of a UV/H₂O₂/ozone neutralization system that has an external hydrogen peroxide and ozone supply 204.

FIG. 3 is a block diagram of an embodiment of the hydrogen peroxide supply 204A that includes ozone.

FIG. 4 is a block diagram of another embodiment of the hydrogen peroxide supply 204B that includes ozone.

FIG. 5. Illustrates the pathways for pathogen and chemical toxin destruction and free radical generating chemical reactions that take place in the UV/H₂O₂/ozone neutralization system.

FIG. 6 is a block diagram of the UV/H₂O₂/ozone neutralization system used in Example 2.

FIG. 7 A-F are photographs of plates that were exposed to air going into and out of the flow-through reaction chamber in experiments designed to test the ability of the UV/H₂O₂/ozone neutralization system to neutralize a large excess of airborne Bacillus globii spores in real time.

FIG. 8. A-F are photographs of plates that were exposed to air going into and out of the flow-through reaction chamber in experiments designed to test the ability of the UV/H₂O₂ neutralization system to neutralize a large excess of airborne Bacillus globii spores in real time without ozone.

### DEFINITIONS

Free radical means intermediate chemical species that contain unpaired electrons, including hydroxyl ions (OH⁻).

Pathogen means any disease-causing organism including bacteria in the vegetative or spore form, viruses, molds, fingi, and parasites. Pathogen also means bacterial toxins as defined herein, prions and biological weapons as defined herein.

Pathogen-neutralized air means air in which the pathogens have been reduced neutralized, inactivated, mutated or killed so that they can no longer reproduce or cause infection.

Decontaminated air means air that has passed through various embodiments of the neutralization systems of the present invention.

Biological weapons means any pathogen, including those listed herein and also their DNA or RNA or fragments, that could be introduced into the air or water or put on surfaces.

Bacterial toxin means any part of a bacterium that is toxic to an animal, including a human.

### DETAILED DESCRIPTION

A method and apparatus are described for neutralizing airborne pathogens and chemical toxins in real time in circulating air. In the disclosed embodiments the pathogens and toxins react with free radical hydroxyl ions (OH⁻) generated by irradiating aqueous hydrogen peroxide (H₂O₂) with broad spectrum, intense UV light in a closed, flow through system, hereafter “the UV/H₂O₂ system.” The UV/H₂O₂ system is designed for use in ventilated air and in heating or air conditioning systems that circulate potentially contaminated air. The embodiments are effective against airborne pathogens including bacteria, viruses, spores, fungi, molds and parasites. The system is also effective in oxidizing airborne chemical toxins, thereby converting potentially deadly compounds to environmentally and physiologically benign compounds. Hydrogen peroxide can be used as the sole source of free radicals or it can be used together with ozone, which also generates highly reactive free radicals upon irradiation with UV light in a moist environment. When ozone is used with H₂O₂, the system is referred to as “the UV/H₂O₂/ozone system.” Various combinations of UV, H₂O₂ and ozone may be advantageous, depending on the targeted pathogens and chemicals. The invention is not limited to the described embodiments. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

The destructive action of ozone dissolved in water on microorganisms is known, particularly on the Escherichia coli (E. Coli), Cryptospondium, Poliovirus and Giardia cysts (including Giardia muris and Giardia lamblia). E. Katzenelson and H. I. Shuval, “Studies on the disinfection of water by ozone: viruses and bacteria”, First International Symposium on Ozone for Water & Wastewater Treatment, Vol. 1, Rice, R. G., and Browning, M. E., Eds., Hampson Press, Washington D.C. (1973); W. T. Broadwater, R. C. Hoehn, and P. H. King, “Sensitivity of three selected bacterial species to ozone”, Appl. Microb. 26:391-393 (1973).

Ozone and UV light have been used either alone or in combination to neutralize pathogens and chemicals in contaminated water (wastewater and water contaminated with industrial pollution). U.S. Pat. No. 4,156,652, 652, U.S. Pat. No. 4,179,616, U.S. Pat. No. 4,204, U.S. Pat. No. 4,230,571. More recently, the combination of UV, ozone and hydrogen peroxide has been used in wastewater purification. Clarin, J. et al., “UV/Ozone/Peroxide Treatment”, published as part of the curriculum for ENVE 436, Civil and Environmental Engineering Department, California Polytechnic State University (hereafter “Clarin”); Jensen, L., U.S. Patent Application No. 2002/0071793; Peyton, G. R., U.S. Pat. No. 5,762,808; Mausgrover et al., U.S. Pat. No. 5,427,693; Zeff et al., U.S. Pat. No. 4,849,114; and Murphy et al., U.S. Pat. No. 6,387,241 B1. Clarin, et al. also reports the use of UV and H₂O₂ without ozone for sterilizing contaminated water. All of these methods require long residence times to accomplish water sterilization. Murphy et al. further describe the use of free radicals generated primarily from ozone and hydrogen peroxide to sterilize soil or contaminants on instruments in a closed chamber, however, very long residence times measured in hours are again required.

In a study aimed at neutralizing pathogens on surfaces, it was shown that ozone in the presence of water vapor neutralizes cultured E. coli and Staphylococcus aureus bacteria on the surface of a petri dish. However, this experiment was conducted in a closed system where ozone was present in concentrations from between 300 and 1500 ppm (part per million) and exposure times were from 10-480 seconds in duration. These conditions therefore do not simulate a situation such as biological warfare where airborne pathogens have been released into a room or a building. Moreover, pathogen neutralization was not achieved in real time, the chamber contained a small volume of stagnant air, and the ozone concentrations were very high. J. Kowalski, W. P. Bahnfleth, and T. S. Whittam, Bactericidal Effects of High Airborne Ozone Concentrations on Escherichia coli and Staphylococcus aureus, Ozone Science & Engineering 20:205-221 (1998). Kowalski, et al. suggested adding UV light to the system to increase toxicity of the ozone, however, this was not tested. The extremely high ozone levels used and the long residence times in the system are unacceptable for real time disinfection of pathogen-contaminated air.

In a related application, Potember discloses a UV/ozone airborne pathogen neutralization system that kills airborne pathogens in real time as the pathogens move with contaminated air through air handling systems into which the system is installed. The pathogens are killed primarily by highly reactive ozone intermediates and free radicals such as hydroxyl ions, which oxidize critical components in the pathogens; UV light and ozone also have intrinsic antimicrobial effects. The system has a flow-through reaction chamber with a chamber air inlet to admit moving pathogen-contaminated air, and a chamber air outlet to release pathogen-neutralized air. A space is defined between the chamber air inlet and outlet that accommodates the passage of moving air through the system. The reaction chamber also contains one or more UV light sources that emit high intensity, broad-spectrum UV light from about 250-280 nanometers (nm). In this system, water is sprayed into the reaction chamber into which ozone gas is introduced. In the moist environment, ozone reacts with UV light to form free radicals and other intermediates that oxidize airborne pathogens thereby neutralizing them.

This UV/ozone system is effective against a wide range of pathogens including Bacillus globii spores, vegetative Erwinia herbicola and MS2 virus. The intermediates and free radicals, generated in the UV/ozone system from the interaction of ozone, water and broad spectrum UV light are more effective at neutralizing pathogens than either ozone or UV light alone, which themselves have intrinsic anti-microbial activity. The UV/ozone neutralization system reduced the extremely high level of airborne vegetative Erwinia herbicola bacteria and bacteriophage MS2 virus introduced into the system to undetectable levels. It also killed a very high percentage of airborne Bacillus globii bacteria spores, which are a simulant for Anthrax. Various embodiments of the UV/ozone system in Potember killed or neutralized very high levels airborne pathogens, and even more importantly, accomplished this in real time in large volumes of moving air. However, while highly effective in killing very large amounts of airborne Bacillus globii bacteria spores, the system was not able to eliminate these spores entirely. Bacterial spores are particularly difficult to destroy because they can survive indefinite periods of starvation, desiccation and exposure to toxic chemicals. Resistance is due to the spore being surrounded by two substantial structures: (1) the cell wall keeps a spore interior dry, thus preventing damage to DNA, and (2) a coating of 20 proteins shields the spore from harmful molecules. A neutralization technology must both penetrate the outer cell wall and destroy critical components of the cell to cause death of the cell or prevent reproduction.

In order to try to increase the efficacy of the UV/ozone pathogen neutralization system against airborne Bacillus globii spores and other pathogens, a concentration of about 25% aqueous hydrogen peroxide was introduced into the reaction chamber together with ozone. Aqueous hydrogen peroxide irradiated by high intensity, broad spectrum UV light from about 150 nm to about 300 nm, particularly from about 250 nm to about 270 nm) generates highly reactive hydroxyl ions (OH⁻) that are powerful oxidizing agents. Whereas the original UV/ozone neutralization system killed or neutralized only about one to two orders of magnitude of a large excess of incoming airborne Bacillus globii spores, the addition of 25% aqueous hydrogen peroxide to ozone and UV light reduced the number of airborne spores leaving the system to undetectable levels. This embodiment of the neutralization system including hydrogen peroxide, ozone and UV light (hereafter referred to as “the UV/H₂O₂/ozone system”) is more effective against airborne bacterial spores than the Potember system. These results are discussed in Example 2.

Additional experiments were performed to test the efficacy of an even lower concentration of 15% hydrogen peroxide and UV light on airborne Bacillus globii spores, without ozone. It was discovered that the system was able to reduce the level of airborne Bacillus globii bacterial spores to undetectable levels in real time as the contaminated air circulated through the reaction chamber at speeds encountered in a typical HVAC system, using 15% aqueous hydrogen peroxide (H₂O₂) and UV light alone. These results are reported in Example 3. This embodiment of the neutralization system, hereafter “the UV/H₂O₂ system,” was as effective as the more complicated UV/H₂O₂/ozone system against airborne spores, thus permitting the construction of simpler systems that do not require ozone. Experiments have been conducted that neutralize a large excess of Bacillus globii spores to undetectable levels in real time in the UV/H₂O₂ system using as little as 3% aqueous hydrogen peroxide. The present embodiments of the UV/hydrogen peroxide systems neutralize pathogens (or chemical toxins which are discussed below) using UV light and from about 0.5% to about 50% aqueous hydrogen peroxide, particularly from about 1% to about 15%.

Hydroxyl radicals generated by the reaction of hydrogen peroxide and UV light, though extremely reactive, have a half-life of approximately 10⁻⁹ seconds, therefore producing no harmful byproducts. Embodiments of the present invention require no chemical reagents, incorporate commercially available components, and can be readily integrated into commercial HVAC systems.

Free radical hydroxyl ions are believed to destroy microorganisms by oxidizing constituent elements of the cell walls before penetrating the microorganisms where they oxidize certain essential elements (e.g., enzymes, proteins, DNA, RNA). When a large part of the membrane barrier is destroyed, the cells lyse (unbind) resulting in immediate neutralization. Other microorganisms, including viruses, are also destroyed by oxidation. Viral DNA is also susceptible to destruction by UV light. It is expected that the free radicals in the present UV/H₂O₂ system will destroy any airborne pathogens including bacteria, viruses, fungi, molds, prions and parasites. Airborne bacterial toxins or fragments of toxic bacterial cell walls will also be neutralized by the UV/hydrogen peroxide system.

Biological agents that can be destroyed using embodiments of the UV/H₂O₂ system or the UV/H₂O₂/ozone system described herein, and the corresponding diseases caused by them, are listed in Table 1. Category A lists high-priority agents including organisms that pose a risk to national security because: they can be easily disseminated or transmitted from person to person; result in high mortality rates and have the potential for major public health impact; might cause public panic and social disruption; and require special action for public health preparedness. Category B lists the second highest priority agents including those that: are moderately easy to disseminate; result in moderate morbidity rates and low mortality rates; and require specific enhancements to the Center for Disease Control's diagnostic capacity and enhanced disease surveillance. Category C lists the third highest priority agents including emerging pathogens that: could be engineered for mass dissemination in the future because of availability; ease of production and dissemination; and potential for high morbidity and mortality rates and major health impact.

TABLE 1
Category A

Anthrax (Bacillus anthracis)
Botulism (Clostridium botulinum toxin)
Plague (Yersinia pestis)
Smallpox (variola major)
Tularemia (Francisella tularensis)
Viral hemorrhagic fevers (filoviruses [e.g., Ebola, Marburg] and
arenaviruses [e.g., Lassa, Machupo])

Category B

Brucellosis (Brucella species)
Epsilon toxin of Clostridium perfringens
Food safety threats (e.g., Salmonella species, Escherichia coli O157:H7,
Shigella)
Glanders (Burkholderia mallei)
Melioidosis (Burkholderia pseudomallei)
Psittacosis (Chlamydia psittaci)
Q fever (Coxiella burnetii)
Ricin toxin from Ricinus communis (castor beans) NEW!
Staphylococcal enterotoxin B
Typhus fever (Rickettsia prowazekii)
Viral encephalitis (alphaviruses [e.g., Venezuelan equine encephalitis,
eastern equine encephalitis, western equine encephalitis])
Water safety threats (e.g., Vibrio cholerae, Cryptosporidium parvum)

Category C

Emerging infectious disease threats such as Nipah virus and hantavirus

1. Structural Components

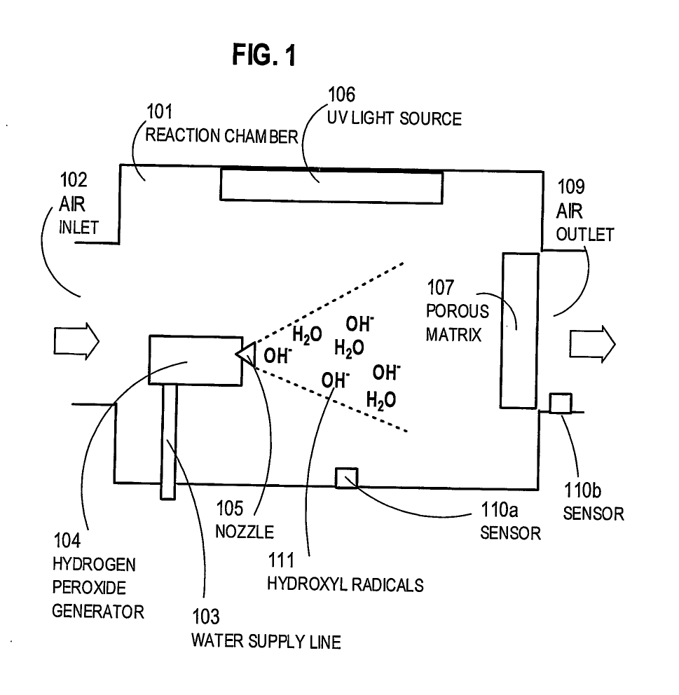

FIG. 1 is an embodiment of a self-contained UV/H₂O₂ system. This embodiment has a flow-through reaction chamber 101 that has a chamber air inlet 102 to admit pathogen-contaminated air, and a chamber air outlet 109 to release pathogen-neutralized air. A space is defined between the chamber air inlet and outlet that accommodates the passage of moving air through the reaction chamber. Reaction chamber 101 contains one or more UV light sources 106 that emit high intensity, broad-spectrum V light. The size of the reaction chamber, the dimensions of the air inlet and air outlet, and the number of UV lights can be varied depending on the volume of air moving through the system.

The reaction chamber 101 contains a hydrogen peroxide generator 104 connected to water supply line 103. Aqueous H₂O₂ produced by hydrogen peroxide generator 104 passes through nozzle 105 and into reaction chamber 101 as a spray, fine mist or vapor. The aqueous H₂O₂ reacts with the UV light provided by UV light source 106 to form hydroxyl free radicals (OH⁻) 111. In some embodiments, the reaction chamber is lined with an UV reflective coating or is built of an UV reflective material.

In another embodiment of the UV/H₂O₂ system, an aqueous H₂O₂ supply (a tank or reservoir or generator) is disposed outside reaction chamber 101, and connected by a supply line to a nozzle disposed inside the reaction chamber 101. If the H₂O₂ in the supply is the desired strength, it is sprayed directly into the reaction chamber through a nozzle. The settings on the nozzle can be adjusted to spray the aqueous H₂O₂ as a spray, mist or vapor. Any means known in the art for introducing aqueous hydrogen peroxide into the reaction chamber can be used.

If the aqueous H₂O₂ in the supply is more concentrated than desired, it can be diluted by mixing with more water before it is sprayed into the reaction chamber using any method for mixing fluids known in the art. In one embodiment, the dilution is accomplished by simply adding the correct amount of water to the aqueous H₂O₂ in the supply tank to obtain the desired strength before turning the system on. In some such embodiments water is provided by self-contained portable water that would make the system suitable for installation in a tank or ambulance. In another embodiment, aqueous hydrogen peroxide is released from the aqueous hydrogen peroxide supply through a conduit and is injected into a mixing bowl. Water is released from a water supply through a conduit and is also injected into the same mixing bowl where it dilutes the hydrogen peroxide to the desired concentration. Methods known in the art for mixing fluids and monitoring the concentration of hydrogen peroxide can be used to automate the system. After the correct concentration of aqueous hydrogen peroxide is achieved, it is introduced as a spray, mist or vapor through a nozzle into the reaction chamber where it is irradiated with UV light to form hydroxyl radicals that kill or neutralize pathogens by oxidation.

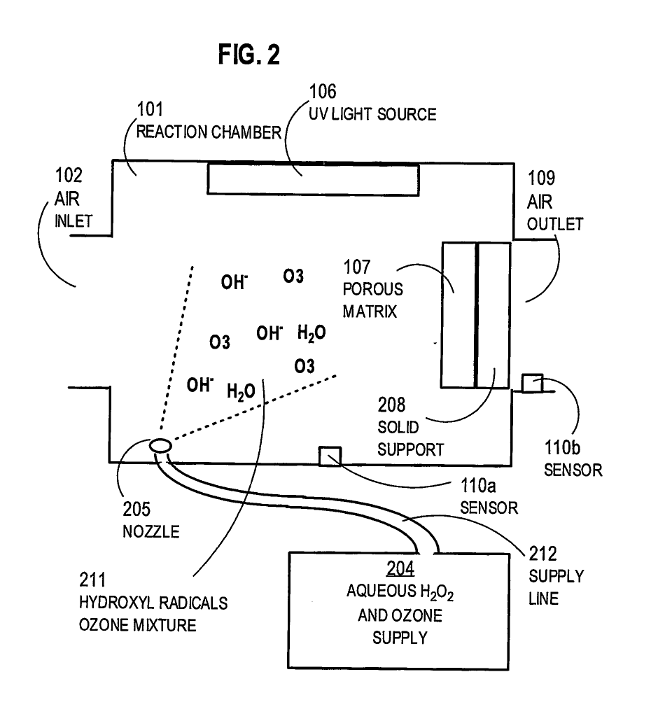

In the illustrated embodiments, an optional porous matrix 107 (FIG. 1 and FIG. 2), such as metal foam, is installed in the reaction chamber to provide additional surface area on which free radicals can react with the pathogens. In one embodiment, the porous matrix covers the reaction chamber air outlet 109 to assure that air passes through the porous matrix before leaving the neutralization system. The porous matrix is recommended where large volumes of air are being decontaminated; its size and thickness can be adjusted to accommodate large volumes of air. The embodiment illustrated in FIG. 1 or FIG. 2 has sensors 110a, 110b to monitor H₂O₂ levels, humidity, temperature, ultraviolet light levels or any other parameter of interest or some combination of these. The sensors can be located inside the reaction chamber 110a, and outside the reaction chamber 110b at a point near air outlet 109. In some embodiments the neutralization system is fully automated so that hydrogen peroxide or ozone or both are dispensed based on measurements obtained from sensors 110a or 110b or both.

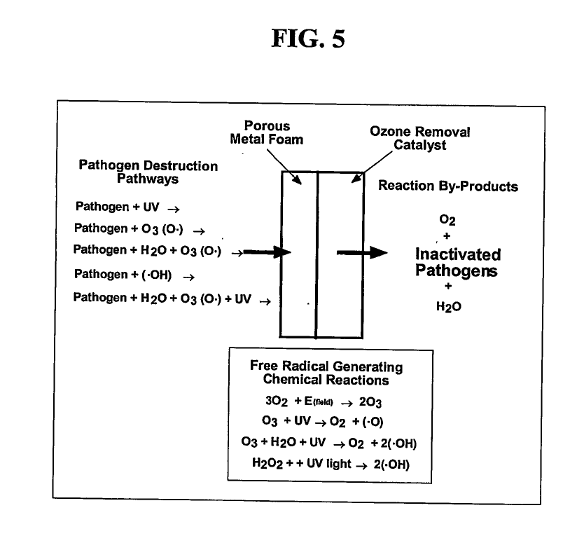

Although the results in Example 3 show that ozone is not required neutralize airborne Bacillus globii spores, it is possible that certain airborne pathogens are more effectively neutralized with the addition of ozone to hydrogen peroxide. UV, hydrogen peroxide, UV light, and ozone are themselves antimicrobial sterilizing agents. Irradiation with high intensity, broad spectrum UV light from about 150 nm to about 300 mnm, especially from about 250 nm to about 270 μm, causes aqueous H₂O₂ and ozone in water to form hydroxyl radicals (OH⁻) that are even more effective pathogen neutralizing agents. The various chemical reactions and free radicals formed in a UV/H₂O₂/ozone system are shown in FIG. 5.

Destruction of airborne chemical toxins by free radical oxidation or by interaction with UV light, ozone, and H₂O₂ or some combination, is accomplished by embodiments of the invention and is discussed in more detail below. Ozone can be generated by a corona discharge generator or by UV light and air, or it can be stored in a tank or reservoir. Ozone can also be generated by ultraviolet light and air. Ozone generators that are compatible with the pathogen-toxin neutralization system of the present invention are described Potember. The ozone supplies and generators are inside the reaction chamber in some embodiments.

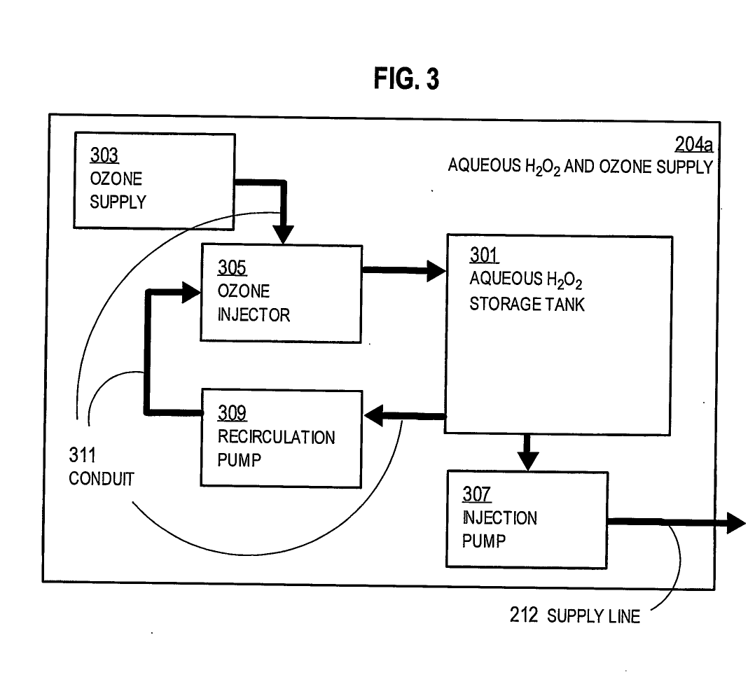

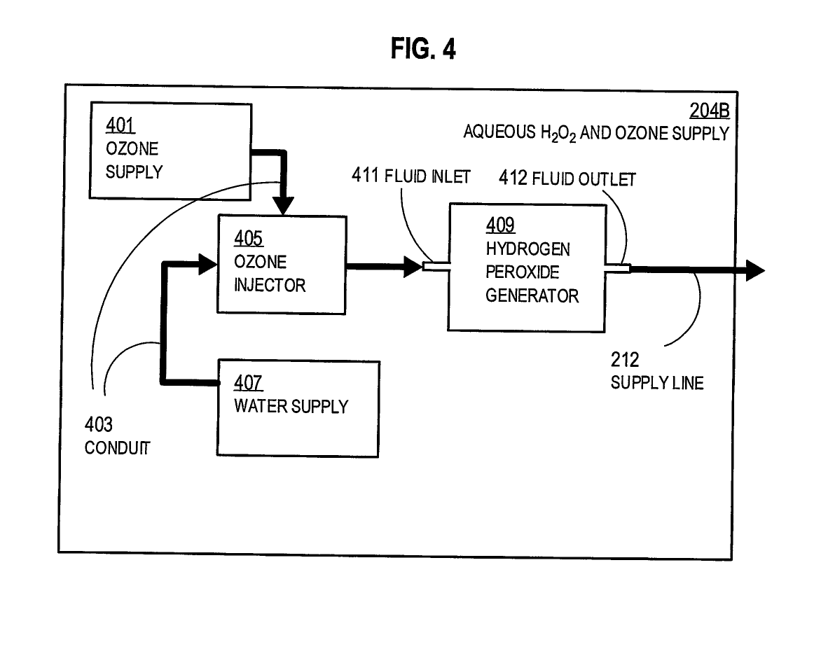

FIG. 2. In the illustrated embodiments of the UV/H₂O₂/ozone system, ozone and aqueous hydrogen peroxide are mixed together before being sprayed into the reaction chamber. An external aqueous hydrogen peroxide and ozone supply combination 204 is provided. Details of different embodiments of the hydrogen peroxide and ozone supply 204 are illustrated in FIG. 3 as 204a, and in FIG. 4 as 204b.

FIG. 3 shows an embodiment of the UV/H₂O₂/ozone system wherein supply 204a has an aqueous H₂O₂ storage tank 301, an ozone generator 303, an injection pump 307, a supply line 212, and conduits 311. Aqueous hydrogen peroxide in storage tank 301 is mixed with ozone supplied by ozone generator 303 when the system is operational. Ozone generated by ozone generator 303 passes through a conduit 311 to ozone injector 305, which injects ozone into aqueous H₂O₂ storage tank 301 to form a mixture of aqueous H₂O₂ and ozone. The mixture thus formed passes through conduit 311 to injection pump 307 that injects the mixture into supply line 212, which is the conduit to reaction chamber 101. Return to FIG. 2. Once the mixture is sprayed into reaction chamber 101 through nozzle 205, the UV light 106 causes H₂O₂, water and ozone to form hydroxyl radicals (OH⁻) 211. Ozone also forms some other highly reactive intermediates illustrated in FIG. 5. Hydroxyl radicals, ozone intermediates, ozone, hydrogen peroxide and UV light mix with and oxidize airborne pathogens in the incoming contaminated air and on porous matrix 107 inside reaction chamber 101, thereby neutralizing the pathogens. Return to FIG. 3. When the UV/H₂O₂/ozone system is on, some of the mixture of hydrogen peroxide, water and ozone in the supply 204a passes through conduit 311 to recirculation pump 309, which pumps the mixture through conduit 311 to ozone injector 305 so that ozone is continually replenished.

Another embodiment of the aqueous H₂O₂ and ozone supply 204 is illustrated in detail as 204b in FIG. 4. In this embodiment the aqueous H₂O₂ and ozone supply 204b is reagent-less, having both an ozone generator 401 and a hydrogen peroxide generator 409. Ozone generated from ozone generator 401 flows through conduit 403 to ozone injector 405 where it is mixed with water. Water flows from water supply 407 through conduit 403 to ozone injector 405. In various embodiments the water supply can be a storage tank or a line hooked up to a building supply of water. The water-ozone mixture flows from ozone injector 405 through conduit 403 to the fluid inlet 411 and into hydrogen peroxide generator 409. Hydrogen peroxide is generated from electricity and water. The mixture of ozone, water and hydrogen peroxide thus formed flows out of hydrogen peroxide generator 409 through fluid outlet 412 and into supply line 212, which is the conduit to the reaction chamber 101. The fluid in the aqueous H₂O₂ and ozone supply in 204a and 204b, or variations thereof, can be moved by fluid pumps or injectors located as needed throughout in the system according to methods known in the art. In some embodiments ozone supply 401 and injector 405 are omitted to create a UV/hydrogen peroxide system.

Ozone generally occurs in natural settings at around 0.02 ppm (parts per million), but it can be found as concentrated as 0.10 ppm, at which level it keeps pathogens in check without being harmful to animals or man. Prolonged exposure to much higher levels of ozone may lead to discomfort, headache, and coughing, warning humans to leave the space and seek better air. OSHA has stipulated that the safe allowable level of residual ozone is 0.1 ppm for continuous exposure throughout an entire 8-hour day for 5 days a week. As soon as ozone is formed in the generator and introduced into the reaction chamber, it either begins to decay back into stable oxygen, or it reacts with water in the presence of high intensity, broad spectrum UV light to form highly active, short-lived intermediates. The maximum half-life of ozone is approximately 30 minutes. However, in practice the half-life is usually much shorter due to interactions with contaminants in the air and contact with surfaces such as walls and carpets. Exposure to ozone levels four to five times the approved levels for short periods of time have no adverse effects because the ozone itself decays back to oxygen rapidly. Levels of ozone from about 0.01 ppm to about 1000 ppm, especially from about 0.1 ppm to about 100 ppm can be maintained in the reaction chamber of the various embodiments of the present neutralization systems.

The embodiment of the UV/H₂O₂/ozone system in FIG. 2 also has an optional solid support 208 made of or coated with one or more agents that adsorb, trap or chemically neutralize airborne pathogens or chemical toxins or both. In some embodiments the solid support is disposed near or next to air outlet 109. The coatings on the solid support can be different depending on the pathogen(s) or chemical toxin(s) being targeted. In the UV/H₂O₂/ozone system, a solid support 208 is included that is coated with or made of ozone removal catalysts and disposed near the air outlet to capture and neutralize any unused ozone before it leaves the system. In some embodiments, more than one solid support is included in the system, each coated with various compounds chosen based on their ability to trap or neutralize the targeted pathogens, or chemical toxins or ozone.

Ozone removal catalysts known in the art include platinum-alumina water vapor catalyst (H₂O—Pt—Al₂O₃) called Dash-220, which decomposes ozone in moist conditions. Other ozone decomposition catalysts include: Corotec Corp. NOZONE® all-aluminum canister; Hoechst Corp. NoXon polymer; a carbon supported metal oxide catalyst (MnO₂—Fe₂O₃, MnO₂); CuCl₂-coated carbon fibers; carbon-iron aerosol particles; alumina; and metal catalysts such as platinum, palladium, and nickel. In one embodiment, the solid support is removable and can be changed when the catalysts have been used up. In another embodiment, the solid support itself is reusable and can be recharged with fresh ozone removal catalysts or other additives or coatings, including antibodies, before being reintroduced into the pathogen-toxin neutralization system.

In various embodiments, the present neutralization system can be operated in continuous or intermittent modes at a wide range of ambient temperatures, including in air cooled by air conditioning or heated in the winter, in desert air that is dry and hot, or very cold air, or some combination. In some embodiments, the chamber is heated by the installation of heating coils that can be disposed on the outside of the chamber, or in the chamber walls. Similarly, in some embodiments the reaction chamber is cooled using any known technology; such as with a cooling tower or cooling coils that remove heat from the neutralization system. In some embodiments a HEPA filter is disposed upstream from the neutralization system to remove approximately 99.97% of airborne particulate matter before contaminated air entered the neutralization system. HEPA filters have an additional important use in that they remove spores that are known to be especially difficult to neutralize in circulating air. However, HEPA filters do not capture viruses. In various embodiments activated carbon filters are used to remove particulate matter; they are disposed either upstream or downstream from the neutralization system, or in both locations.

In some embodiments, the reaction chamber has more than one chamber air inlet or outlet or both. This permits the installation of the neutralization system at locations where several ducts converge. In other embodiments the neutralization system is entirely self-contained. In some entirely self-contained systems, the hydrogen peroxide generator is disposed inside the reaction chamber. The water supply is connected to a tank. Thus, the neutralization system can be scaled down to a size that is portable, and suitable for use in vehicles such as military tanks.

The present neutralization systems can also be used to clean air circulating through air conditioning or heating systems having one or more ducts that move and direct the circulating air. It can be installed in existing heating and air conditioning ducts by removing a section of the existing duct to accommodate the reaction chamber, and connecting the reaction chamber to the existing duct at the chamber air inlet and outlet. The various embodiments of the neutralization systems of the present invention are installed so that contaminated air passes into the chamber from the existing duct through the air inlet, and decontaminated pathogen-neutralized air leaves the system through the air outlet from which it passes back into the existing duct for recirculation. To assure that the contaminated air enters and passes through the neutralization system, the chamber air inlet and outlet are adapted to fit the existing ducts using methods known in the art so that no air is allowed to bypass the system. In one embodiment, the chamber air inlet/outlet is adapted to fit an existing building air duct using a flange, with a rubber O-ring between the chamber wall and the flange to prevent air leaks.

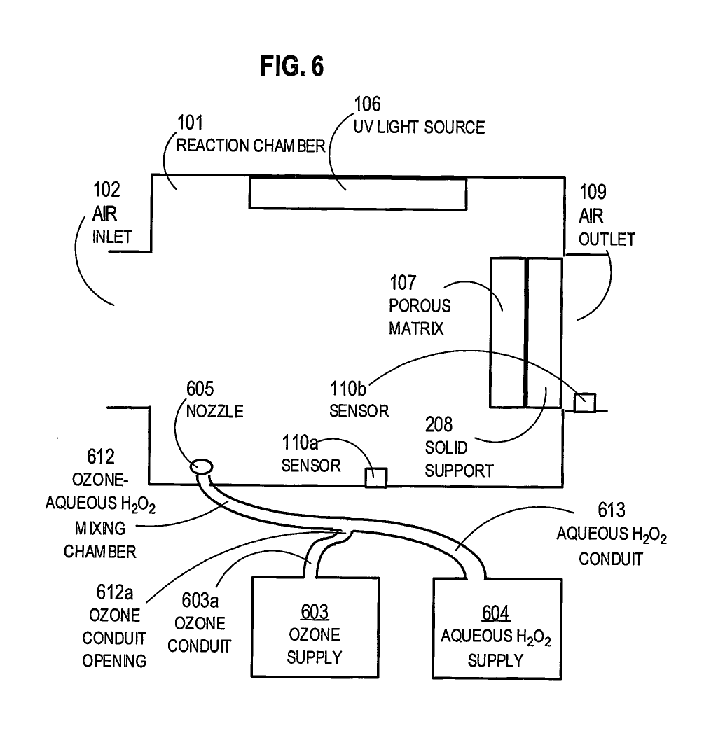

FIG. 6 illustrates the embodiment the UV/H₂O₂/ozone system that was used in Example 2. Ozone supply 603 is an ozone gas generator connected to ozone conduit 603a, which connects to ozone-aqueous H₂O₂ mixing chamber 612 at ozone conduit opening 612a. Downstream from the ozone generator is an aqueous hydrogen peroxide supply 604 that releases a stream of aqueous hydrogen peroxide into aqueous hydrogen peroxide conduit 613. As the aqueous hydrogen peroxide stream flows past ozone conduit opening 612a, it creates a vacuum that helps to pull the ozone exiting the ozone generator 603 through ozone conduit 603a into the ozone-aqueous H₂O₂ mixing chamber 612 at conduit opening 612a, where a mixture of ozone and aqueous H₂O₂ is formed. After the ozone/aqueous hydrogen peroxide mixture is formed, it is sprayed through nozzle 605 into reaction chamber 101. It is then irradiated with UV light to form hydroxyl radicals and other reactive intermediates that kill pathogens (and neutralize chemical toxins).

2. Methods for Neutralizing Pathogens or Chemical Toxins

Although the steps of the method for neutralizing pathogens using the neutralization system of the present invention are described in a particular order below, in other embodiments the steps may occur in a different order or overlapping in time. An embodiment of a method of neutralizing airborne pathogens or chemical toxins in ventilated air involves the steps of:
a. introducing air contaminated with pathogens or chemical toxins or both into a flow-through reaction chamber;
b. introducing aqueous hydrogen peroxide into the flow-through reaction chamber to form a mixture of contaminated air and aqueous hydrogen peroxide inside the reaction chamber;
c. irradiating the mixture with ultraviolet light thereby neutralizing the airborne pathogens or chemical toxins or both to create decontaminated air; and
d. releasing the decontaminated air from the reaction chamber.

In some embodiments there is an additional step before step c of introducing ozone into the reaction chamber to form a mixture of contaminated air, aqueous hydrogen peroxide and ozone. This mixture is then irradiated to neutralize airborne pathogens or chemical toxins or both. In other embodiments, aqueous hydrogen peroxide is mixed with ozone before being introduced into the reaction chamber. Thus a mixture of contaminated air, aqueous hydrogen peroxide and ozone is formed in step b, and this mixture is irradiated in step c. In some embodiments, a concentration of hydrogen peroxide is maintained in the flow through reaction chamber at a level in a range from about 1% to about 50%, especially from about 1% to about 25%. In embodiments where ozone is introduced, a concentration of ozone in the reaction chamber is maintained at a level in a range from about 0.01 ppm to about 100 ppm, especially from 0.1 ppm to about 100 ppm.

3. Advantages of the UV/H₂O₂ and the UV/H₂O₂/Ozone Neutralization Systems

Advantages of the illustrated embodiments of the UV/H₂O₂ and the UV/H₂O₂/ozone neutralization systems include the following:
The neutralization systems neutralize airborne pathogens or chemical toxins in real time in large volumes of moving air;
The neutralization systems can be installed in conjunction with other air pathogen-toxin neutralization technologies such as installing the neutralization system at a location that receives air that has been passed through a pre-existing HEPA filter system.
The neutralization systems are activated and operated electrically.
The major components of the neutralization systems are commercially available.
The neutralization systems can be reagent-less by using a hydrogen peroxide or an ozone generator, or both. Hydrogen peroxide can be generated from water using electricity. Water can be provided from a portable supply or from a building's low-pressure supply. Ozone is generated from water and molecular oxygen in the room air.
Stable byproducts are created UV irradiation generates hydroxyl ions that are extremely short-lived. If ozone is used, a solid support coated with ozone removal catalysts prevents unused ozone from exiting the reaction chamber. If any residual aqueous hydrogen peroxide exits the system in decontaminated air, it can be captured using, for example, a dehumidifier downstream from the air outlet of the reaction chamber or any other method for trapping or neutralizing hydrogen peroxide. In another example, additional UV lights could be placed outside the system near the air outlet to convert escaping hydrogen peroxide to very short-lived hydroxyl radicals and water. Residual hydrogen peroxide is expected to be small enough, particularly with concentrations of aqueous hydrogen peroxide below about 25%.
The neutralization system requires minimum maintenance.
One or more UV lights can be operated in tandem or independently with the ozone generator or the hydrogen peroxide generator.
Commercially available hydrogen peroxide, humidity, particle sampling, ozone, and UV light sensors allow the neutralization systems to be microprocessor controlled and continually balanced.
Some embodiments include an open-pore metal foam support in the reaction chamber that produces a beneficial low-pressure drop across the neutralization system. The porous matrix also provides the medium in which concentrations of hydroxyl radicals, hydrogen peroxide, ozone, and ozone intermediates diffuse and react with airborne pathogens or chemical toxins.
Several pathogen- and chemical toxin-neutralization approaches can be combined by the present invention: the UV/H₂O₂ system combines H₂O₂, UV and hydroxyl radical sterilization; the UV/H₂O₂/ozone system combines sterilization by H₂O₂, UV, ozone, hydroxyl radicals and other highly reactive ozone intermediates.
The system is flexible. UV light plus hydrogen peroxide or ozone or a mixture of both at various concentrations can be chosen based on the targeted pathogens and chemical toxins. The use of ozone may not always be needed. Also the solid support and coatings thereon can be selected or changed as needed to adsorb or neutralize various combinations of airborne pathogens and chemicals.

Both the UV/H₂O₂ and the UV/H₂O₂/ozone neutralization systems can be self-contained. For example, in one embodiment, the UV/H₂O₂ system includes a hydrogen peroxide generator or reservoir and a portable re-circulating water supply that permits water to be reused. Various embodiments of the neutralization systems can be made in different sizes that can be adapted for installation in cars, tanks, aircraft, etc. The installation of the present neutralization systems in ventilated air handling system is simple. Installation can be accomplished by cutting an opening in an existing air duct in a structure and removing a section of it to accommodate system. The actual system is then installed in the existing duct by connecting the chamber air inlet and chamber air outlet of the system in sealing relation to the existing duct so that pathogen-contaminated air is blown into the reaction chamber through the chamber air inlet, and decontaminated air leaves the system through the chamber air outlet. The illustrated embodiments require electricity, and some embodiments require a hook up to a water supply.

4. Details of Structural Components

4.1 Hydrogen Peroxide and Ozone Supply

The results set forth in Examples 2 and 3 show that levels of about 15-25% H₂O₂ in water reduced airborne Bacillus globii spores to undetectable levels in real time as they passed through the reaction chamber. Fifteen percent H₂O₂ was chosen because it is safe to handle without burning the skin. The optimum concentration of H₂O₂ may vary depending on the ambient temperature and the targeted pathogens or chemical toxins. H₂O₂ for use in the present system can be generated chemically or electrically using any method known in the art, including those listed in Table 2.

TABLE 2
Chemical generation of H₂O₂:

| | | | |
|---|---|---|---|
| | Catalyst/anthroquinone | | |
| O₂ + H₂ | = | H₂O₂ | Commercial preparation |

Electrical generation of H₂O₂:

| | | |
|---|---|---|
| H₂O + Electricity = | H₂O₂ | Electrochemical generation |

Free radical (OH⁻) generating chemical Reactions based on H₂O₂

| | | |
|---|---|---|
| H₂O₂ + Quaternary ammonium salts | = | H₂O + OH⁻ Chemical generation |
| H₂O + H₂O₂ | = | H₂O + OH⁻ hydroxyl radicals |
| | Catalyst | |
| H₂O + H₂O₂ | = | H₂O + OH⁻ hydroxyl radicals |
| | UV light | |
| H₂O + H₂O₂ | = | H₂O+ OH⁻ hydroxyl radicals |
| | Heat | |

Use of a hydrogen peroxide generator has the advantage that it is reagent-less, thus eliminating the need to store or transport chemicals. The use of an H₂O₂ generator permits the design of a lighter neutralization system that can be easily transported into remote locations where limited resources are available. Only water and electricity need to be supplied. Thus, the UV/H₂O₂ neutralization system can be scaled down to a size that is portable, and suitable for use in vehicles such as military tanks.

Electrochemical generation of H₂O₂ is accomplished by passing water over a stacked electrode bank. One such electrode bank is made of parallel Titanium-metal plates coated with titanium oxide (TiO₂) doped with 4-mole % nibidium (n) or tantalum (t) in the +4 oxidation state. This oxide coating is heavily n-doped which makes the system extremely corrosion resistant at the potential (+2.74 V) required to generate the radical (OH⁻). This method has demonstrated hydroxyl radical generation at 7.5-19 moles/kilowatts per hour when operated in a continuous flow mode for other applications. The hydrogen peroxide generator releases hydrogen peroxide on demand to maintain desired levels inside the reaction chamber. There is no limit on the concentration of hydrogen peroxide that can be used in the present systems, however, amounts ranging from about 1% to about 50%, or more typically from about 3% to about 25%, are sufficient to neutralize most airborne pathogens or chemical toxins or combinations thereof. Routine experimentation as described in the examples under controlled conditions with known agents, biological or chemical, or combinations thereof will demonstrate the optimum levels of H₂O₂ or ozone. For some uses, the level of aqueous hydrogen peroxide in the system may be sufficient at less than 1%. The level of H₂O₂ supplied to the system can be monitored by one or more sensors. Any method for producing H₂O₂ can be used in the present invention.

To optimize pathogen or chemical toxin neutralization, the pH of the water introduced into the system can be adjusted. Routine experimentation will determine the optimum pH for neutralizing various pathogens, chemical toxins or combinations thereof. Where neutralization of a given pathogen is enhanced by acidic pH, the water can be treated with acetic acid to obtain the desired pH before it is sprayed into the neutralization system. Alternatively, a basic pH can be obtained where beneficial. In some embodiments, a coating is applied to the porous matrix or solid support that adjusts the pH to a level that optimizes neutralization of the targeted pathogen or chemical toxin.

4.2 Broad Spectrum UV Light Source

UV light from about 100 nm to about 400 nm causes hydrogen peroxide to form highly active free radical intermediates that in turn react with and destroy pathogens. An UV wavelength of about 250 nm to about 270 was used in the examples. UV radiation is itself intrinsically toxic to some pathogens, causing radiation damage to the pathogen's DNA so that it cannot reproduce. High levels of UV radiation are considered lethal for most microorganisms, including bacteria, fungal spores, viruses, protozoa, nematode eggs and algae. That part of the UV light spectrum known to kill or neutralize most pathogens is between 100-400 nm, which is just shorter than the wavelengths of visible light. However, UV sterilization is more effective on surfaces than on airborne pathogens. Bacteria are the easiest pathogens to neutralize; viruses and spores are more resistant. Spores of the Bacillus species possess a thick protein coat that consists of an electron-dense outer coat layer and a lamella-like inner coat layer. This coating reduces the effect of UV irradiation on the pathogen's DNA.

Incandescent, quartz or mercury vapor lamps are suitable for use in the present pathogen-toxin neutralization systems. UV light can be continuous or pulsed, and high intensity UV lights are preferred. In a flashing UV light, each high power flash or pulse lasts only a few hundred millionths of a second. A typical flash of UV light lasts from about 1 to about one millionth of a second, and has a flash repetition rate from about 1 to 10 flashes per second. The duration, wavelength, and intensity of the UV light can be adjusted to optimize the effect on various pathogens. Flash frequency can vary from 1-1000 per second as determined by experimentation.

The use of UV light is the easiest way to convert H₂O₂ to hydroxyl radicals, and ozone to hydroxyl radicals and other reactive intermediates, however any method known in the art can be incorporated into the present system in addition to or in place of UV lights, including the use of heat and catalysts.

4.3 Porous Matrix Composition

In some embodiments a porous matrix disposed inside the reaction chamber provides an increased surface area on which the free radicals, ozone intermediates, ozone and hydrogen peroxide contact and react with airborne pathogens in a micro-solvent environment. The solvent is water that condenses on the pathogens. In the examples, the porous matrix used was a DUCOCEL® aluminum metal foam having a pore size of 40 PPI (pores per square inch) and 8% density. DUCOCEL® is used to mix rocket fuels prior to ignition. It has a reticulated structure of open, duodecahedronal-shaped cells connected by continuous, solid metal ligaments. The matrix is completely repeatable, regular and uniform throughout the material. DUCOCEL® is a rigid, highly porous and permeable structure with a controlled density of metal per unit volume that does not reduce the flow rate of incoming air. It is available in 6101 and A356 aluminum alloys and in vitreous or glassy carbon, other metals, ceramics and composite materials. Density is continuously variable from 3-12 percent. No degradation of DUCOCEL® was seen after several months of use. The DUCOCEL® matrix adds a large surface area on which the ozone intermediates and pathogens interact. The matrix does not noticeably impede the airflow. In some embodiments, the porous matrix is removable and reusable. In various embodiments, the volume, thickness and density of the porous matrix can be varied depending on the volume of contaminated air being passed through the neutralization system and the size of the chamber air outlet. Porous matrices from 1 inch to 3 inches thick have been tested and do not impede air flow.

Increasing the air velocity serves to mix the contaminated incoming air with the water vapor containing highly reactive free radicals. In some embodiments where very large volumes of air are being decontaminated, one or more fans are added to the system to further assist mixing contaminated air with the free radicals, ozone, hydrogen peroxide and water. In embodiment for large-scale applications, the airflow is dramatically increased by increasing the cross sectional area of the porous metal structure and adjusting the size of the air inlets and air outlets.

Any solid porous matrix can be used that increases surface area without blocking air outflow from the neutralization system or inhibiting the formation of the highly reactive ozone intermediates. In some embodiments, metal foams that have antibacterial activity are used, such as copper and silver. Porous matrices of plastics, polymers, particle balls, thread or ceramics or some combination thereof is also used in various embodiments. In some embodiments, the porous matrix is coated with one or more non-volatile antibacterial, antiviral and antispore agents that increase pathogen-toxin adsorption and/or neutralization. This may be advantageous where a pathogen is highly resistant to neutralization. In various embodiments the matrix or one or more solid supports is coated with agents that trap and/or neutralize chemical weapons. In some embodiments the coatings are biological or chemical or both, and include compounds such as antibodies directed to bacterial cell walls, DNA, bacterial toxins, viruses, prions, etc. Routine experimentation will determine which additives are the most effective, and this will vary depending on the targeted pathogens or toxins. Similarly, coatings that adsorb or neutralize chemical toxins can be put onto one or more solid supports in the various systems of the present invention.

4.4 Use of Surfactants, Ultrasound, and Microwaves

To increase the effectiveness of ozone, hydrogen peroxide and free radicals on airborne pathogens, especially spores, nontoxic surfactants (soap molecules) are pre-mixed with the water and sprayed into the reaction chamber in some embodiments. It is expected that the surfactants increase the contact time between ozone and ozone free radicals and pathogens, thus facilitating pathogen-toxin neutralization. One or more nontoxic surfactants known in the art, or mixtures thereof, can be used in various embodiments.

Any means of disrupting or fracturing the pathogens, including the coating protecting spores, increases the effectiveness of neutralization in the embodiments. It is expected that the disruption facilitates the interaction of the highly active free-radicals, hydrogen peroxide, ozone intermediates, free ozone and UV light to interact with the pathogen. Microwaves and/or ultrasound may help to break down the spore coating to make the spores more susceptible to neutralization. Plasma DC glow discharge has been shown to be an effective sterilization method for medical devices on its own. The principle sterilization using plasma DC glow discharge is intense UV radiation in the 160-240 nm range. Therefore in some embodiments, the neutralization system further includes a plasma DC glow discharge UV tube, a microwave generator, or an ultrasound generator or some combination. In alternative embodiments, contaminated air is treated before it enters the system by placing a means for producing microwave irradiation, plasma DC glow discharge, and/or ultrasound upstream near the chamber air inlet.

In some embodiments, aqueous hydrogen peroxide and contaminated air are mixed together, with or without ozone, in a vortex mixer that is irradiated with UV light before being sprayed into the reaction chamber, where it is optionally irradiated again with UV light. In an embodiment, ozone is also added to aqueous hydrogen peroxide and contaminated air. The components are introduced through separate lines into the mixer. In some embodiments aqueous hydrogen peroxide and ozone are premixed before being introduced into the vortex mixer where they are further mixed with incoming contaminated air mixture.

4.5 Chemical Toxin Neutralization

Some embodiments of the UV/H₂O₂ and the UV/H₂O₂/ozone systems are not only useful in neutralizing airborne bacteria, they also destroy airborne chemical weapons by converting the chemical toxins to benign forms, primarily by oxidation. Hydroxyl ions and ozone intermediates are very powerful oxidants that react with organic molecules to form carbon dioxide and water. Chemicals that are known to be oxidized to environmentally benign forms by hydrogen peroxide and hydroxyl ions include: organic compounds, chlorinated VOCs, mercaptans, nitriles, aldehydes, alcohols, amines, metals, alkylboranes, azo-compounds, cyanides, phenols, sulfides, chromium and some inorganics. O'Brien & Gere Engineers, Inc. Innovative Engineering Technologies for Hazardous Waste Remediation, New York: Van Nostrand Reinold; 1995; Clarin et al. UV radiation by itself degrades polychlorinated biphenyls, dioxins, polyaromatic compounds and breaks many covalent bonds. Clarin, et al. It also enhances chemical oxidation, though the mechanism is uncertain. One theory is that organic compounds absorb light energy at visible or UV wavelengths and as a result are easier to destroy.

It has been reported that UV, ozone and peroxide treatment of contaminated water destroys halogenated solvents, phenol, pentachlorophenol, pesticides, polychlorinated biphenyls, explosives, BTEX, MTBE and many other organic compounds. Clarin, et al. Table 4 contains a list of chemical agents. To the extent that any of the compounds named above and in Table 4 become airborne and are oxidized to a benign state by hydroxyl radicals, hydrogen peroxide, UV light or ozone, the embodiments of the present neutralization systems will be effective. Routine experimentation will determine the optimum levels of hydrogen peroxide, ozone, or combinations thereof for neutralizing each pathogen or chemical agent. Some embodiments of the neutralization systems of the present invention include one or more solid supports that are coated with biological or chemical agents that trap or neutralize airborne chemical weapons.

TABLE 4
CHEMICAL AGENTS

| | |
|---|---|
| Abrin | Methyldichloroarsine (MD) |
| Adamsite (DM) | Mustard Gas (H) (Sulfur |
| Agent 15 | Mustard) |
| Ammonia | Mustard/Lewisite (HL) |
| Arsenic | Mustard/T |
| Arsine (SA) | Nitrogen Mustard (HN-1, |
| Benzene | HN-2, HN-3) |
| Bromobenzylcyanide CA | Nitrogen Oxide (NO) |
| BZ | Paraquat |
| Cannabinoids | Perflurorisobutylene (PHIB) |
| Chlorine (CL) | Phenodichloroarsine (PD) |
| Chloroacetophenone (CN) | Phenothiazines |
| Chloropicrin (PS) | Phosgene (CG) |
| CNB (CN in Benzene and | Phosgene Oxime (CX) |
| Carbon Tetrachloride) | Phosphine |
| CNC (CN in Chloroform) | Potassium Cyanide (KCN) |
| CNS (CN and Chloropicrin | Red Phosphorous (RP) |
| in Chloroform) | Ricin |
| CR | Sarin (GB) |
| CS | Sesqui Mustard |
| Cyanogen Chloride (CK) | Sodium Cyanide (NaCN) |
| Cyclohexyl Sarin (GF) | Soman (GD) |
| Diphenylchloroarsine (DA) | Sulfur Mustard (H) (Mustard |
| Diphenylcyanoarsine (DC) | Gas) |
| Diphosgene (DP) | Sulfur Trioxide- |
| Distilled Mustard (HID) | Chlorosulfonic Acid (FS) |
| Ethyldichloroarsine (ED) | Tabun (GA) |
| Fentanyls and Other Opioids | Teflon and |
| Hydrofluoric Acid | Perflurorisobutylene (PHIB) |
| Hydrogen Chloride | Thallium |
| Hydrogen Cyanide (AC) | VX |
| Lewisite (L, L-1, L-2, L-3) | |
| LSD | |

Embodiments of the UV/H₂O₂ system and the UV/H₂O₂/ozone system neutralize pathogens and other toxins in several ways. The initial elements themselves (UV, ozone and hydrogen peroxide) have intrinsic sterilizing potential, and the hydroxyl radicals and ozone intermediates formed upon UV irradiation are yet more reactive than the parent compounds. Ozone, its intermediates and hydroxyl radicals react with organic molecules in many ways, including inserting an oxygen into a benzene ring, breaking double bonds to form aldehydes and ketones, and reacting with alcohol to form organic acids. LaGrega, M. et al., Hazardous Waste Management, New York: McGraw-Hill, Inc. For example, it is known that deadly cyanide (NaCN) is oxidized by ozone to a safer product NaCNO as follows: O₃ NaCN=>NaCNO+O₂.

Embodiments of the neutralization systems described herein are adapted to be portable so that they fit into trucks that transport food to prevent pathogens or chemical toxins in the air from contaminating the food.

In the foregoing specification, the invention has been described with reference to specific embodiments thereof. It will, however, be evident that various modifications and changes may be made to the inventions without departing from the broader spirit and scope of the invention. The efficacy of some embodiments is described in further detail, in the following non-limiting examples.

### 5.0 EXAMPLES

### 5.1 Example 1

### Experimental Set Up

A. The Air Sampler

The New Brunswick Scientific Microbiological Air Sampler Series STA-204 (a slit sampler) was used to test performance of the embodiments. To test the extent of contamination in incoming air just before it enters the neutralization system, samples were passed over the system inlet plate. Samples of outgoing air just after leaving the system were passed over the system outlet plate. The slit sampler works by drawing a known total volume of air by vacuum through a slit opening. A pressure drop that occurs across the slit causes the air with its entrained contaminants to accelerate to a higher velocity. The airborne pathogen contaminants, because of their heavier mass, are impacted onto the surface of a sterile petri dish placed on a rotating, timed turntable. Only the small area of surface of the agar that is disposed just below the slit is exposed to the contaminated air. Thus as the dish rotates, different sectors of agar are exposed. A sample time of thirty minutes was selected for all experiments. The sampler was set so that the duration of the experiment is equivalent to one complete revolution of the petri plate. When the sample time has elapsed, no further air sample is taken. A particle distribution guide can be used to estimate the time at which contamination occurred. The guide is a mylar disk that is divided into thirty segments by lines that emanate from near the center to a marker circle near the outer edge. The bottom of the petri dish is marked with a line to indicate the position of the dish at time zero. This makes it easy to line up the particle guide. In the examples, samples of incoming air taken continuously were impacted onto the system air inlet plate, and samples of outgoing air were impacted onto the System air outlet plate during each thirty minute experiment. The experiments described in the examples using the UV/H₂O₂/ozone system (Example 2) and the UV/H₂O₂ system (Example 3) were conducted in the same way, except that the neutralization system in Example 2 included ozone while the system in Example 3 did not.

In the examples, the systems included a fan to help pull air through the system. The fan was turned on in all experiments. A modified residential air handler was used as the reaction chamber in the examples, that has a nominal capacity of 1000 cubic feet per meter (cfm) through an 18 by 20.5 inch opening. The air inlet was restricted to the size of a 6-inch round duct in the test model, and 2.5×3.25 inch foam metal DUCOCEL® matrix covered the air outlet, which had the same dimension. The air volume passing through the foam metal was only 50 cfm. The air velocity through system is about 78 fpm (normally ˜500 cfm), while the velocity through foam metal is ˜1616 cfm. This occurs because air accelerates as it passes through the system. The smaller, restricted air inlet serves to dramatically increase the air velocity that mixes intake air with pathogens and water spray containing free radical species.

A CD-5 GENESIS™ corona discharge ozone generator 603 made by Del Industries, Inc. with maximum output of 5 g/hr was disposed outside the reaction chamber. The UV light source 106 consisted of two BioAire-UV Lights Model BUV 24DE Double Ended Fixtures. The brand of light is not critical; however, more powerful UV lights are preferred. New pulsed UV light sources that are extremely powerful are available and may be used in the present invention. The size of the reaction chamber was 45 inches length×21 inches height×23 inches diameter. The air inlet 202 and air outlet 209 were sized to fit tightly onto a commercially available flexible duct, to which duct they were connected with a flange or collar and a rubber seal. This tight connection prevents air loss and assures that air leaving the air duct had passed through the UV/ozone neutralization system.

A porous metal foam 107 matrix was made of DUCOCEL® aluminum metal foam having a density of 8% and 40 PPI was used. Several sheets of the foam were cut and stacked until the stack measured 3.5 inches long and two inches in height and thickness. The matrix was held in place by restriction plates and was installed so that it was just in front of and covered the chamber air outlet 109 so that the air that enters the system passes through the matrix before exiting the neutralization system.

Room air entered the neutralization system through the chamber air inlet. The humidity of the disinfected air leaving the reaction chamber varied from about to 65 percent, and the temperature was room temperature. The ozone generator and the UV light source were operated in tandem throughout the experiments, and the neutralization system was operated in a continuous mode with the fan on during the experiments.

The nozzles used put out approximately 29 ml of fluid per minute on the surface of the metal matrix material. Air exiting the system has a humidity of about 55-60%. Bacillus globii spores were cultured in the laboratory using standard techniques well known in the art until they attained a cell density of about 5.3×10⁹ CFU/ml.

B. Introduction of Airborne Pathogens into the Neutralization System.

In each experiment in Examples 2 and 3, Bacillus globii spores were introduced into the reaction chamber using the MICRO MIST™ nebulizer. Bacillus globii spores were cultured in the laboratory using standard techniques well known in the art until they attained a cell density of about 5.3×10⁹ CFU/ml. About 3-6 ml of these cultured bacterial spores (about 3-6×10⁹ spores) was introduced into the reaction chamber in each Test below. No spores were introduced in the control experiments.

### 5.2 Example 2

### The UV/H₂O₂/Ozone System

The parameters of the experiment are set forth in Table 3 below.

TABLE 3

| Parameter Name | Parameter Setting |
|---|---|
| Airflow Rate | 50 CFM |
| Water flow to nozzle | 0.46 GPH (1.74 ml/hr) |
| Injector Model Number | 684 |
| O₃ Concentration | As attained using |
| | injector and flow rate |
| | specified |
| H₂O₂ Concentration | 25% |
| # of UV lights | 6 |
| Position of UV lights | 8″ from media pad |
| Number of Nozzles | 1 |
| Model of Nozzle | 684 |
| Position of Nozzle | 2.5″ from media pad |
| Media Pad Size | 1.5 × 3.25 |
| Velocity Through media pad | 1,600 FPM |

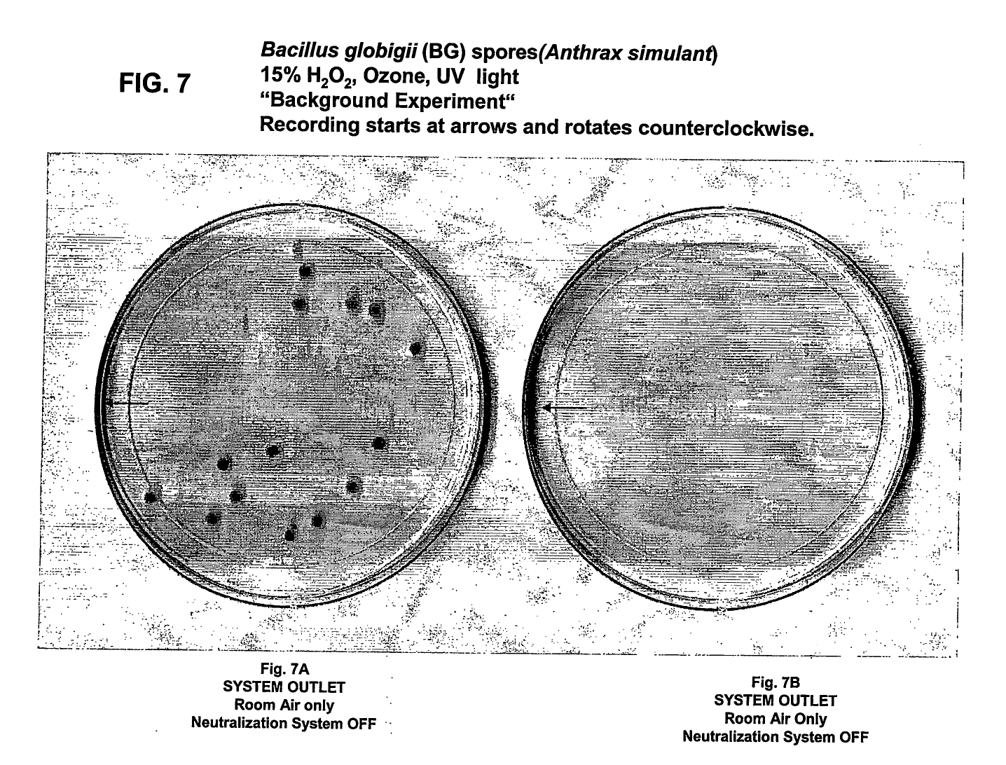

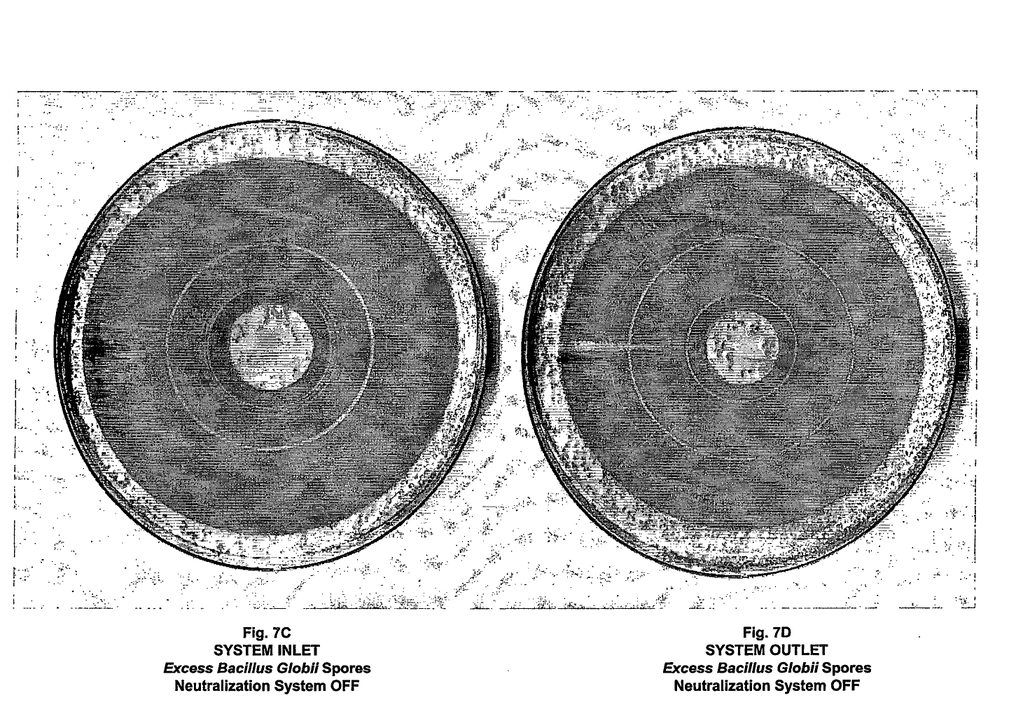

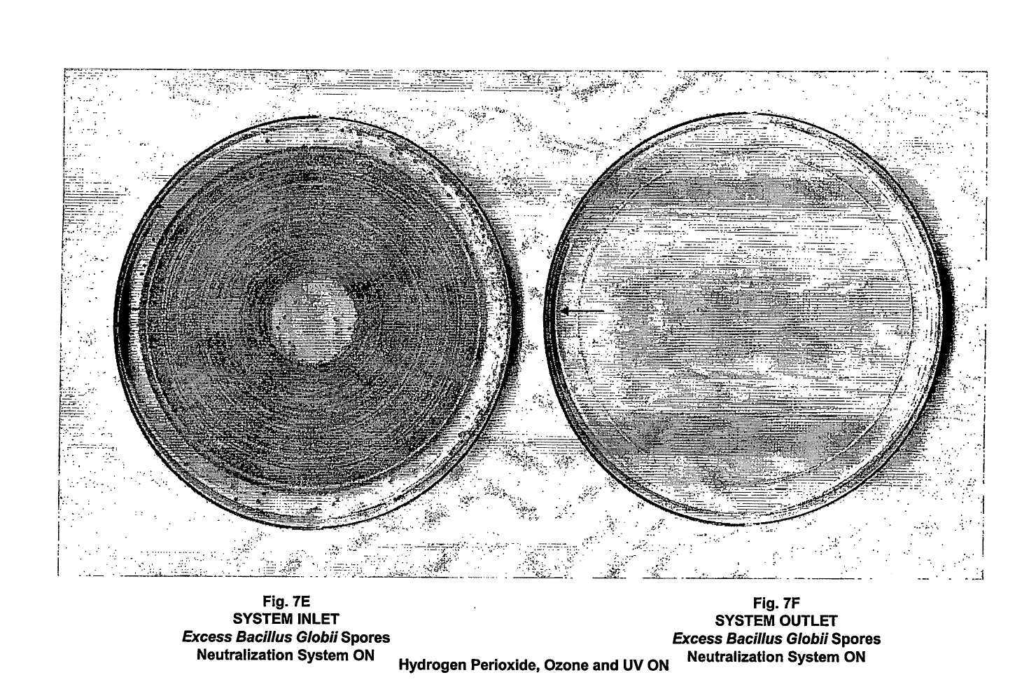

Control: Room air was drawn through an inactive neutralization system with all components switched off before any spores were intentionally introduced. No hydrogen peroxide or ozone was introduced, and the UV light was off. The system inlet plate was exposed to incoming room air before it entered the reaction chamber 101, and the system outlet plate was exposed to outgoing air that had passed through the reaction chamber 101 of the inactivated neutralization system. After a thirty minute exposure, the system inlet and outlet plates were collected and cultured for 24 hours at 37 degrees centigrade. Fourteen colonies were observed on the system inlet plate FIG. 7A, but no colonies were observed on the system outlet plate FIG. 7B. The few colonies on the air inlet plate indicated that the air inlet was not properly cleaned from an earlier experiment, but this did not affect the results of the experiments. The absence of colonies on the system outlet plate shows that there are no bacteria in the reaction chamber 101.

Test 1: Bacillus globii spores were introduced into the reaction chamber air inlet 102 with all systems off (no hydrogen peroxide, no ozone, UV light off). After a thirty minute exposure, the system inlet and outlet plates were collected and cultured for 24 hours at 37 degrees C. Both the system inlet and system outlet plates were overgrown with bacteria, such that the CFU were too numerous to count as shown in FIGS. 7C and 7D.

Test 2: Bacillus globii spores were introduced into the reaction chamber air inlet with the UV/H₂O₂/ozone system fully activated (aqueous H₂O₂ on, Ozone On, UV On). After a thirty minute exposure, the system inlet and outlet plates were collected and cultured for 24 hours at 37 degrees C. As expected, the system inlet plate was overgrown with bacteria with CFU too numerous to count. (FIG. 7E). By contrast, no CFU were counted on the system outlet plate (FIG. 7F). A large excess of airborne Bacillus globii spores deliberately introduced in high numbers and passed through the neutralization system in real time, was thus reduced to undetectable levels by the UV/H₂O₂/ozone system.

### 5.3 Example 3

### The UV/H₂O₂System

The UV/H₂O₂ system used in Example 3 is the same as in FIG. 6, except that the ozone generator was turned off.

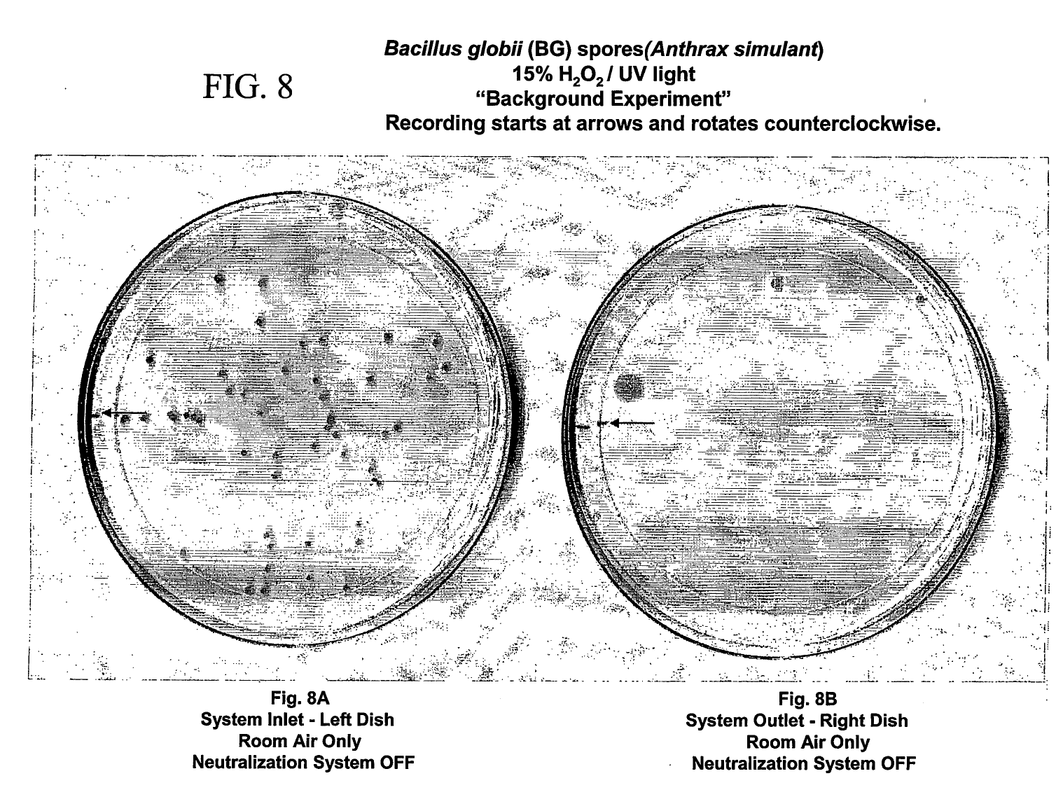

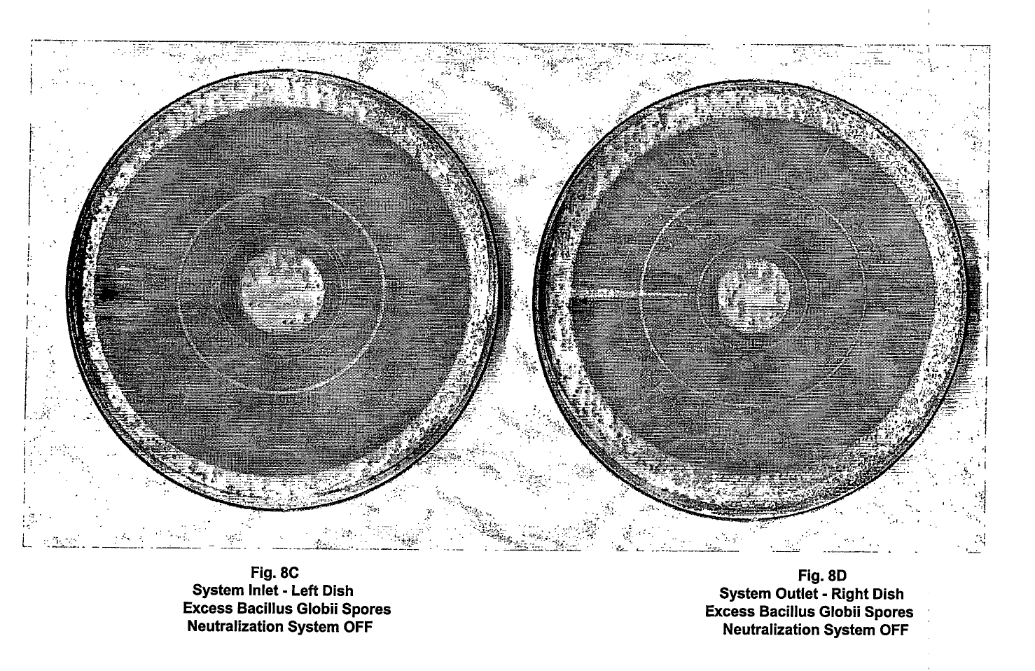

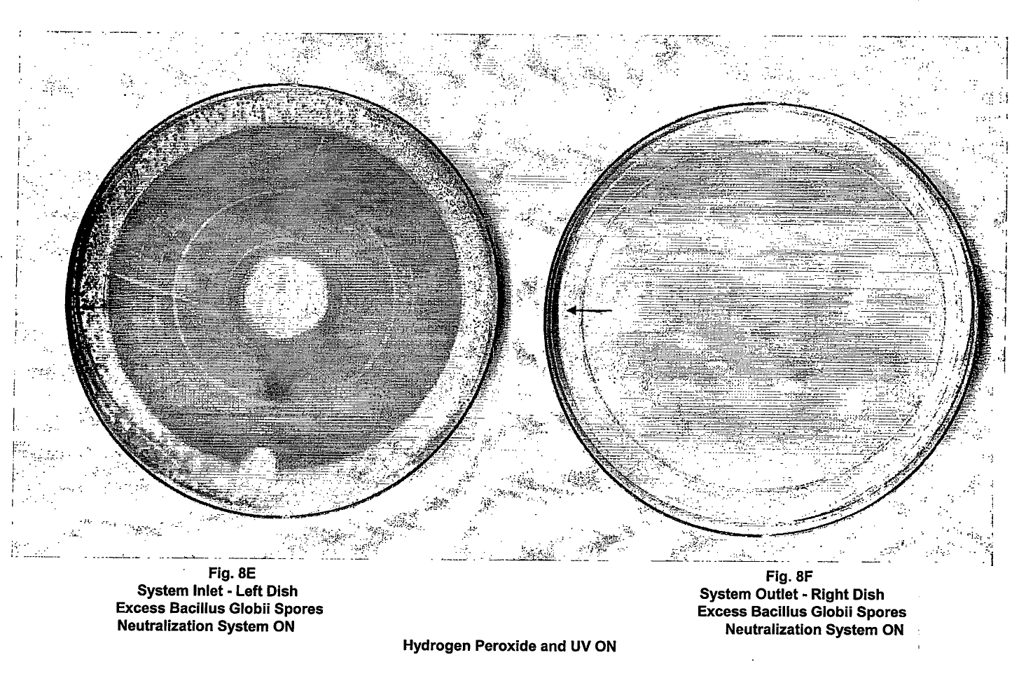

Control: As a control, room air was drawn through an inactive neutralization system with all components switched off. No hydrogen peroxide was introduced and the UV light was off. The system inlet plate was exposed to incoming room air before it entered the reaction chamber 101, and the system outlet plate was exposed to outgoing air that had passed through the reaction chamber 101 of the inactivated neutralization system. After a thirty minute exposure, the system inlet and outlet plates were collected and cultured for 24 hours at 37 degrees C. Fifty-one colonies were counted on the System Inlet Dish. No colonies were observed on the system outlet plate, FIGS. 8A and 8B. The absence of colonies on the system outlet plate shows that there are no bacteria in the reaction chamber 101.

Test 1: Bacillus globii spores were introduced into the reaction chamber air inlet 102 with all systems off (no hydrogen peroxide, UV light off.) After a thirty minute exposure, the system inlet and outlet plates were collected and cultured for 24 hours at 37 degrees C. Both the system inlet and system outlet plates were overgrown with bacteria, such that the CFU were too numerous to count. FIGS. 8C and 8D.

Test 2: Bacillus globii spores were introduced into the reaction chamber air inlet 102 with the UV/H₂O₂ system fully activated (aqueous H₂O₂ on, UV On). After a thirty minute exposure, the system inlet and outlet plates were collected and cultured for 24 hours at 37 degrees C. The CFU on the system inlet plate were too numerous to count (FIG. 8E). By contrast, no CFU were counted on the system outlet plate (FIG. 8F). The large excess of airborne Bacillus globii spores deliberately introduced in high numbers and passed through the neutralization system in real time, were reduced to undetectable levels by 15% UV/H₂O₂ system even without ozone.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

What is claimed is:

1. A system for neutralizing airborne pathogens, comprising:
A. a flow-through reaction chamber having:
1. a chamber air inlet at a first end of the reaction chamber to admit air contaminated with pathogens, and
2. a chamber air outlet at a second end of the reaction chamber to release decontaminated air, and defining between the air inlet and air outlet a passageway,
B. a supply of aqueous hydrogen peroxide connected to a conduit for introducing aqueous hydrogen peroxide into the reaction chamber, and
C. an ultraviolet light source for introducing UV light into the reaction chamber.

2. The system as in claim 1, wherein the supply of aqueous hydrogen peroxide is a hydrogen peroxide generator connected to a water supply and a source of electricity.

3. The system as in claim 1, wherein the supply of aqueous hydrogen peroxide is a reservoir of aqueous hydrogen peroxide.

4. The system as in claim 1, wherein the conduit is a nozzle disposed inside the reaction chamber.

5. The system as in claim 1, wherein the reaction chamber further comprises a porous matrix.

6. The system as in claim 5, wherein the porous matrix is metal foam.

7. The system as in claim 6, wherein the metal is selected from the group comprising aluminum, copper, silver, and oxides thereof.

8. The system as in claim 6, wherein the metal foam is aluminum foam.

9. The system as in claim 5, wherein the porous matrix is removable.

10. The system as in claim 1, further comprising a microwave generator to introduce microwaves into the reaction chamber.

11. The system as in claim 1, further comprising an ultrasonic wave generator to introduce ultrasonic waves into the reaction chamber.

12. The system as in claim 1, further comprising an ozone supply for introducing ozone into the reaction chamber.

13. The system as in claim 12, wherein the ozone supply is an ozone generator.

14. The system as in claim 12, wherein the ozone supply is a reservoir that contains ozone.

15. The system of claim 12, further comprising a mixing chamber for mixing ozone and aqueous hydrogen peroxide.

16. The system of claim 1, wherein the reaction chamber further comprises a solid support.

17. The system of claim 16, wherein the solid support comprises ozone removal catalysts.

18. The system of claim 16, wherein the solid support comprises compounds that adsorb or neutralize pathogens.

19. The system of claim 16, wherein the solid support comprises compounds that adsorb or neutralize chemical toxins.

20. The system of claim 19, wherein the solid support comprises ozone removal catalysts.

21. The system of claim 17, wherein the ozone removal catalyst is a member selected from the group comprising all-aluminum catalysts, a carbon supported metal oxide catalyst, CuCl₂-coated carbon fibers, carbon-iron aerosol particles, alumina, platinum, palladium, and nickel.

22. The system of claim 13, wherein the ozone generator is a corona discharge generator.

23. The system as in claim 1, configured for operation in a continuous mode.

24. The system as in claim 1, configured to be activated upon demand.

25. The system of claim 1, further comprising a fan to move air through the passageway.

26. The system of claim 1, wherein an amount of hydrogen peroxide in the reaction chamber is controlled by sensors.

27. The system as in claim 1, wherein the ultraviolet light source emits high intensity UV light.

28. The system as in claim 27, wherein the ultraviolet light source emits UV light having a wavelength in a range from about 250 nanometers to about 300 nanometers.

29. The system of claim 1, wherein a concentration of hydrogen peroxide in the aqueous hydrogen peroxide supply is from about 1% to about 50%.

30. The system as in claim 1, wherein a concentration of hydrogen peroxide in the aqueous hydrogen peroxide supply is from about 1% to about 25%.

31. A method of neutralizing airborne pathogens comprising:
1. introducing air contaminated with pathogens into a flow-through reaction chamber;
2. introducing aqueous hydrogen peroxide into the flow-through reaction chamber to form a mixture of contaminated air and aqueous hydrogen peroxide inside the reaction chamber;
3. irradiating the mixture with ultraviolet light thereby neutralizing the airborne pathogens to create decontaminated air; and
4. releasing the decontaminated air from the reaction chamber.

32. The method of claim 31, further comprising the additional step before step 3 of introducing ozone into the reaction chamber forming a mixture of contaminated air, aqueous hydrogen peroxide and ozone.

33. The method of claim 31, step 2 further comprising mixing the aqueous hydrogen peroxide with ozone before introducing the aqueous hydrogen peroxide to form a mixture of contaminated air, aqueous hydrogen peroxide and ozone.

34. The method of claim 31, step 2 further comprising introducing the aqueous hydrogen peroxide into the reaction chamber through a nozzle disposed in the reaction chamber, to form at least one of a spray, mist or vapor.

35. The method as in claim 31, step 2 further comprising maintaining a concentration of hydrogen peroxide in the flow through reaction chamber at a level in a range from about 1% to about 50%.

36. The system as in claim 31, step 2 further comprising maintaining a concentration of hydrogen peroxide in the flow-through reaction chamber at a level in a range from about 1% to about 25%.

37. The method as in claim 32, step 2 further comprising maintaining a concentration of ozone in the reaction chamber at a level in a range from about 0.01 ppm to about 100 ppm.

38. A method of neutralizing airborne chemical toxins comprising:
1. introducing air contaminated with chemical toxins into a flow-through reaction chamber;
2. introducing aqueous hydrogen peroxide into the flow-through reaction chamber to form a mixture of contaminated air and aqueous hydrogen peroxide inside the reaction chamber;
3. irradiating the mixture with ultraviolet light thereby neutralizing the airborne chemical toxins to create decontaminated air; and
4. releasing the decontaminated air from the reaction chamber.

39. The method of claim 38, further comprising the additional step before step 3 of introducing ozone into the reaction chamber to form a mixture of contaminated air, aqueous hydrogen peroxide and ozone.

40. The method of claim 38, step 2 further comprising mixing the aqueous hydrogen peroxide with ozone before introducing the aqueous hydrogen peroxide to form a mixture of contaminated air, aqueous hydrogen peroxide and ozone.

41. The method of claim 38, step 2 further comprising introducing the aqueous hydrogen peroxide into the reaction chamber through a nozzle to form at least one of a spray, mist or vapor.

42. The method as in claim 38, step 2 further comprising maintaining a concentration of hydrogen peroxide in the flow through reaction chamber at a level in a range from about 1% to about 50%.

43. The system as in claim 38, step 2 further comprising maintaining a concentration of hydrogen peroxide in the flow-through reaction chamber at a level in a range from about 1% to about 25%.

44. The method as in claim 32 or claim 33, step 2 further comprising maintaining a concentration of ozone in the reaction chamber at a level in a range from about 0.01 ppm to about 1000 ppm.

45. The method as in claim 32 or claim 33, step 2 further comprising maintaining a concentration of ozone in the reaction chamber at a level in a range from about 0.01 ppm to about 1000 ppm.

46. A system for neutralizing airborne pathogens and chemical toxins, comprising:
A. a flow-through reaction chamber having:
1. a chamber air inlet at a first end of the reaction chamber to admit air contaminated with pathogens, and
3. a chamber air outlet at a second end of the reaction chamber to release decontaminated air, and defining between the air inlet and air outlet a passageway,
B. a supply of aqueous hydrogen peroxide connected to a conduit for introducing aqueous hydrogen peroxide into the reaction chamber, and
C. a means for converting aqueous hydrogen peroxide to hydroxyl radicals.

47. The system as in claim 46, wherein the means for converting aqueous hydrogen peroxide into hydroxyl radicals is heat.

48. The system as in claim 46, wherein the means for converting aqueous hydrogen peroxide into hydroxyl radicals is electricity.
